(19) United States
(12) Patent Application Publication
(10) Pub. No.: US 2025/0012711 A1
(43) Pub. Date: Jan. 9, 2025

(54) SELF-CALIBRATING DIAGNOSTIC DEVICE AND SYSTEMS AND METHODS FOR USE THEREOF

(71) Applicant: LABRADOR SCIENCES LTD., Tel Aviv (IL)

(72) Inventors: Yoav KESSLER, Tel Aviv (IL); Amit AVNER, Tel Aviv (IL)

(21) Appl. No.: 18/711,824

(22) PCT Filed: Nov. 23, 2022
(86) PCT No.: PCT/IL2022/051245

§ 371 Date: May 20, 2024

Related U.S. Application Data
(60) Provisional application No. 63/282,223, filed on Nov. 23, 2021.

(51) Int. Cl. G01N 21/27; G01N 21/78

(52) U.S. Cl. CPC G01N 21/274; G01N 21/78

(57) ABSTRACT

A method for obtaining calibrated indicia of analyte levels in a sample including introducing a test sample possibly containing at least one analyte into a testing device, processing the test sample, obtaining a measurable indication of a level of the at least one analyte in the test sample, processing at least one control sample containing the at least one analyte in the device, the processing of the control sample being carried out in at least near real time with respect to the processing of the test sample, obtaining at least one measurable indication of a level of the at least one analyte in the control sample and calibrating the indication of the level of the analyte(s) in the test sample based on the indication of the level of the analyte(s) in the control sample, to provide a calibrated indication of the level of the analyte(s) in the test sample.

### RELATED APPLICATIONS

Reference is hereby made to U.S. Provisional Patent Application No. 63/282,223, entitled ‘SELF-CALIBRATING DIAGNOSTIC DEVICE AND SYSTEMS AND METHODS FOR USE THEREOF’, filed Nov. 23, 2021, the disclosure of which is hereby incorporated by reference and priority of which is hereby claimed pursuant to 37 CFR 1.78(a)(4) and (5)(i).

### FIELD OF THE INVENTION

The present invention relates generally to medical devices and more particularly to in-vitro diagnostic medical devices having self-calibrating functionality.

### BACKGROUND OF THE INVENTION

Various types of diagnostic devices and systems and methods for calibration of diagnostic measurements, are known in the art.

### SUMMARY OF THE INVENTION

The present invention seeks to provide novel in-vitro medical devices having highly accurate self-calibrating capabilities, as well as systems and methods for employing such devices.

There is thus provided in accordance with a preferred embodiment of the present invention a method for obtaining calibrated indicia of a level of at least one analyte in a sample including introducing a test sample possibly containing at least one analyte into a testing device, processing the test sample in a test region of the testing device, obtaining, as a result of the processing, a measurable indication of a level of the at least one analyte in the test sample, processing at least one control sample containing the at least one analyte in a control region of the testing device, the processing of the at least one control sample being carried out in at least near real time with respect to the processing of the test sample, obtaining, as a result of the processing of the control sample, at least one measurable indication of at least one level of the at least one analyte in the at least one control sample and calibrating the indication of the level of the at least one analyte in the test sample based on the at least one measurable indication of the level of the at least one analyte in the at least one control sample, to provide a calibrated indication of the level of the at least one analyte in the test sample.

Preferably, the control sample is separate to and different from the test sample.

Preferably, the test sample includes a bodily fluid obtained from a subject.

Preferably, the testing device is a single-use, disposable device.

Preferably, the measurable indication of the level of the at least one analyte in the test sample and the control sample includes at least one of a colorimetric indication, an optical indication, an electrical indication and a chemical indication.

Preferably, the calibrating includes at least one of qualitative calibrating based on relative characteristics of the measurable indications of the test sample and control sample, and quantitative calibrating based on relative concentrations of the at least one analyte as derived from the measurable indications of the test sample and control sample.

Preferably, the at least one control sample includes at least two control samples having mutually different concentrations of the analyte therein and the quantitative calibrating includes finding a correlation between the different concentrations of the analyte in the at least two control samples and the measurable indications thereof, and applying the correlation to the measurable indication of the level of the analyte in the test sample.

Preferably the method also includes, following the calibrating, rating a level of the at least one analyte in the test sample based on the calibrating, and providing a diagnosis of a subject from whom the test sample is obtained based on the rated level of the at least one analyte in the test sample.

Preferably, the providing a diagnosis includes providing one of a binary diagnosis and a probability of the subject having the diagnosis.

Preferably, the calibrating is at least partially carried out by processing functionality within at least one of the testing device and the cloud.

There is also provided in accordance with another preferred embodiment of the present invention a system for diagnosing a subject including a testing device configured to receive a test sample from a subject, the test sample possibly containing at least one analyte, process the test sample in a test region of the testing device, output, as a result of the processing, a measurable indication of a level of the at least one analyte in the test sample; process, in at least near real time with respect to the processing of the test sample, at least one control sample containing the at least one analyte, in a control region of the testing device; and output, as a result of the processing of the at least one control sample, a measurable indication of a level of the at least one analyte in the control sample, an image acquisition device operative to capture the measurable indications of the levels of the at least one analyte in the test sample and the at least one control sample, and a data analysis module operative to calibrate the captured measurable indication of the level of the at least one analyte in the test sample based on the captured measurable indication of the level of the at least one analyte in the at least one control sample and to output a diagnosis of the subject based on the calibrated level of the at least one analyte in the test sample.

Preferably, in the system the control sample is separate to and different from the test sample.

Preferably, in the system the test sample includes a bodily fluid obtained from the subject.

Preferably, the testing device of the system is a single-use, disposable device.

Preferably, in the system the measurable indication of the level of the at least one analyte in the test sample and the control sample includes at least one of a colorimetric indication, an optical indication, an electrical indication and a chemical indication.

Preferably, the data analysis module is operative to perform at least one of qualitative calibration based on relative characteristics of the measurable indications of the test sample and control sample, and quantitative calibration based on relative concentrations of the at least one analyte as derived from the measurable indications of the test sample and control sample.

Preferably, in the system the at least one control sample includes at least two control samples having mutually different concentrations of the analyte therein and the data analysis module is operative to find a correlation between the different concentrations of the analyte in the at least two control samples and the measurable indications thereof, and to apply the correlation to the measurable indication of the level of the analyte in the test sample in order to derive the quantitative calibration.

Preferably, the data analysis module is also operative to rate a level of the at least one analyte in the test sample based on the calibration, and provide the diagnosis of the subject from whom the test sample is obtained based on the rated level of the at least one analyte in the test sample.

Preferably, the diagnosis provided by the system includes one of a binary diagnosis and a probability of the subject having the diagnosis.

Preferably, the data analysis module is at least partially incorporated within at least one of the testing device and processing functionality in the cloud.

There is also provided in accordance with a further embodiment of the present invention a testing device including a test region configured to process therein a test sample obtained from a subject, a first output region configured to display a measurable indication of a level of at least one analyte in the test sample, a control region configured to process therein at least one control sample, the control sample being processed in at least near real time with respect to the test sample and a second output region configured to display, in at least near real time with respect to the display of the measurable indication of the level of the at least one analyte in the test sample, a measurable indication of at least one level of the at least one analyte in the at least one control sample, the measurable indication of the at least one level of the at least one analyte in the at least one control sample providing a basis for calibration of the measurable indication of the level of the at least one analyte in the test sample.

Preferably, the control sample of the device is separate to and different from the test sample.

Preferably, the test sample of the device includes a bodily fluid obtained from a subject.

Preferably, the testing device is a single-use, disposable device.

Preferably, in the device, the measurable indication of the level of the at least one analyte in the test sample and the control sample includes at least one of a colorimetric indication, an optical indication, an electrical indication and a chemical indication.

Preferably, in the device the calibration includes at least one of qualitative calibration based on relative characteristics of the measurable indications of the test sample and control sample, and quantitative calibration based on relative concentrations of the at least one analyte as derived from the measurable indications of the test sample and control sample.

Preferably, in the device the at least one control sample includes at least two control samples having mutually different concentrations of the analyte therein and the quantitative calibration includes finding a correlation between the different concentrations of the analyte in the at least two control samples and the measurable indications thereof, and applying the correlation to the measurable indication of the level of the analyte in the test sample.

Preferably, the device also includes, at least partially incorporated therein, data analysis functionality operative to rate a level of the at least one analyte in the test sample based on the calibrating, and provide a diagnosis of a subject from whom the test sample is obtained based on the rated level of the at least one analyte in the test sample.

Preferably, the diagnosis provided by the device includes one of a binary diagnosis and a probability of the subject having the diagnosis.

Preferably, in the device, the calibration is at least partially carried out by at least one of the data analysis functionality and processing functionality in the cloud.

There is further provided in accordance with yet another preferred embodiment of the present invention a system for diagnosing a subject including a testing device including a test sample receipt subsystem operative to receive a test sample from a subject, the test sample possibly containing at least one analyte, a first processing subsystem operative to process the test sample in a test region of the testing device, a first data output subsystem operative to output, as a result of the processing by the first processing subsystem, a measurable indication of a level of the at least one analyte in the test sample, a second processing subsystem operative to process, in at least near real time with respect to the processing of the test sample by the first processing subsystem, at least one control sample containing the least one analyte, in a control region of the testing device and a second data output subsystem operative to output, as a result of the processing of the control sample, a measurable indication of at least one level of the at least one analyte in the at least one control sample, an image acquisition device operative to capture the measurable indications of the levels of the at least one analyte in the test sample and the at least one control sample, and a data analysis module operative to calibrate the captured measurable indication of the level of the at least one analyte in the test sample based on the captured measurable indication of the at least one level of the at least one analyte in the at least one control sample and to output a diagnosis of the subject based on the calibrated level of the at least one analyte in the test sample.

Preferably, the control sample of the system is separate to and different from the test sample.

Preferably, the test sample of the system is a bodily fluid obtained from the subject.

Preferably, the testing device of the system is a single-use, disposable device.

Preferably, the measurable indication of the level of the at least one analyte in the test sample and the control sample includes at least one of a colorimetric indication, an optical indication, an electrical indication and a chemical indication.

Preferably, the data analysis module is operative to perform at least one of qualitative calibration based on relative characteristics of the measurable indications of the test sample and control sample, and quantitative calibration based on relative concentrations of the at least one analyte as derived from the measurable indications of the test sample and control sample.

Preferably, the at least one control sample includes at least two control samples having mutually different concentrations of the analyte therein and the data analysis module is operative to find a correlation between the different concentrations of the analyte in the at least two control samples and the measurable indications thereof, and to apply the correlation to the measurable indication of the level of the analyte in the test sample in order to derive the quantitative calibration.

Preferably, the data analysis module is also operative to rate a level of the at least one analyte in the test sample based on the calibration, and provide the diagnosis of the subject from whom the test sample is obtained based on the rated level of the at least one analyte in the test sample.

Preferably, the diagnosis includes one of a binary diagnosis and a probability of the subject having the diagnosis.

Preferably, the data analysis module is at least partially incorporated within at least one of the testing device and processing functionality in the cloud.

### BRIEF DESCRIPTION OF THE DRAWINGS

The present invention will be understood and appreciated more fully based on the following detailed description taken in conjunction with the drawings, in which:

FIG. 1 is a simplified partially pictorial, partially block diagram illustration of a system including a self-calibrating diagnostic device, constructed and operative in accordance with a preferred embodiment of the present invention;

FIG. 2 is a simplified flow chart illustrating steps in the operation of a diagnostic device of the type within the system of FIG. 1;

FIGS. 3A-3D are simplified exemplary outputs of a diagnostic device of the type of FIGS. 1 and 2;

FIG. 4 is a simplified schematic illustration of an algorithm for analyzing outputs of a diagnostic device, such as outputs shown in FIGS. 3A-3D; and

FIG. 5 is a simplified flow chart diagram illustrating a method for obtaining calibrated indicia of a level of an analyte in a sample, in accordance with another preferred embodiment of the present invention.

### DETAILED DESCRIPTION OF PREFERRED EMBODIMENTS

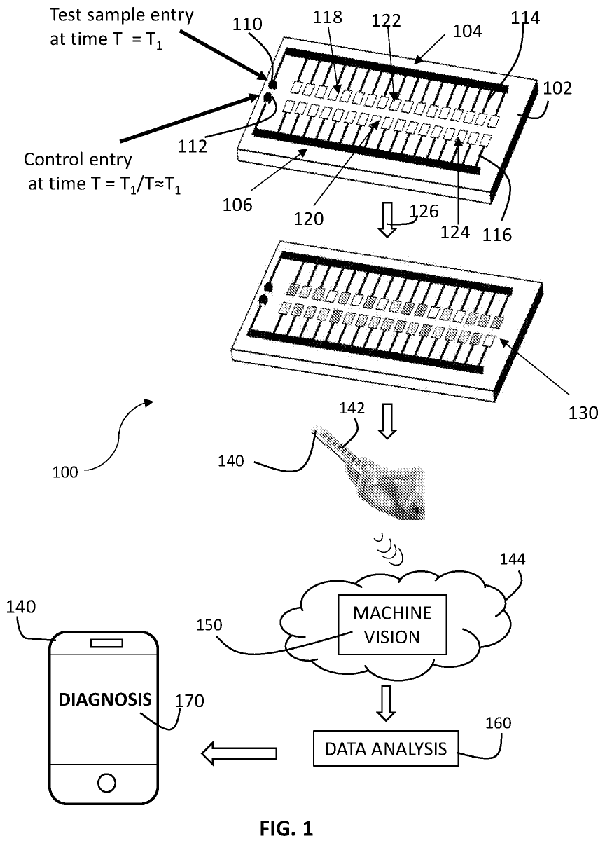

Reference is now made to FIG. 1, which is a simplified partially pictorial, partially block diagram illustration of a system including a self-calibrating diagnostic device, constructed and operative in accordance with a preferred embodiment of the present invention.

As seen in FIG. 1, there is provided a system 100, preferably for monitoring or diagnosis of a medical condition of a subject. System 100 preferably includes a medical device, preferably embodied as a diagnostic device 102. Diagnostic device 102 is an in-vitro diagnostic device, intended for use by the subject themselves or by a healthcare provider assisting the subject. As will be appreciated from the description henceforth, diagnostic device 102 is simple and straightforward to use, such that diagnostic device 102 is well suited for use by a non-medical professional, for example for home use by a subject. Diagnostic device 102 is preferably, although not necessarily, a single use, disposable device.

Preferred embodiments of the present invention, such as system 100 and diagnostic device 102 forming a part thereof, relate to the testing of at least one control sample in real time, or near real time, with respect to the testing of a test sample obtained from a subject. The at least one control sample is preferably separate from and different to the test sample and may be a synthetic substance synthesized so as to have properties that imitate the natural properties of the test sample. A measurable indication of a level of at least one analyte in the at least one control sample provides a reference based on which a measurable indication of a level of the at least one analyte in the test sample may be calibrated. The calibrated indication of the test sample may then be used to ascertain the presence and absolute or relative levels of one or more analytes in the test sample and hence diagnose a condition of the subject from which the test sample was obtained.

By way of example, the measurable indications of analyte levels in the test and control samples may be in the form of colorimetric signatures, or may be in the form of other measurable indications, which may be electrical, optical, chemical or any other form of measurable indications. It is appreciated that system 100 of the present invention may be applied to outputs of a variety of types of measurement assays, including, but not limited to, electrochemical, biochemical and optical assays.

While it is appreciated that the system, device and method of the present invention may be employed with respect to any suitable test and control samples providing any type of measurable output indication of analyte levels therein, the test sample being obtained from a human or non-human subject, the system, device and method of the present invention are particularly suitable for use in colorimetric saliva testing. Accordingly, much of the description which follows relates to the use of the present invention in the context of colorimetric saliva testing. Colorimetric saliva testing may be, for example, for the purpose of detecting and diagnosing one or more of Type I diabetes, Type II diabetes, pre-diabetes, periodontitis or other diseases in the subject from which the saliva is obtained. It is understood that in the following description the terms ‘diagnosing’ and ‘detecting’ are used interchangeably when describing the diagnostic output of systems, devices and methods of the present invention.

It is appreciated that, in the context of colorimetric saliva testing for diabetes detection as well as other types of colorimetric testing of a sample for the presence and level of one or more analytes therein, the colorimetric test results may be highly sensitive to environmental conditions. For example, colorimetric signatures may vary significantly according to ambient temperature, humidity or other conditions. Furthermore, in the case of comparing colorimetric signatures of a test sample to those of a control for calibration purposes, colors of the colorimetric signature of the control may be difficult to accurately replicate per test result, making use of a standardized colorimetric control signature difficult. Additionally, manufacturing tolerances of in-vitro medical devices may cause significant deviation between results obtained and displayed by different devices.

In order to overcome these difficulties, as well as provide other advantages as will be apparent from this description, the system, method and device of the present invention involve calibration of colorimetric analysis of saliva for the presence of one or more analytes therein, with respect to colorimetric analysis of a control sample that is processed in at least near real time with respect to the processing of the saliva. The processing of the control sample in at least near real time, and particularly preferably simultaneously, with respect to the processing of the saliva, ensures that the control and test sample share identical or near identical testing conditions, such that the colorimetric outputs of each are subject to the same or substantially the same environmental factors.

Furthermore, the processing of a control sample per test sample obviates the need for attempted reproduction of colors of the control sample results, ensuring that colorimetric output of the test sample is compared to actual colorimetric output of the control sample. In this way, the control serves as a highly accurate standard based on which the colorimetric test results of the test sample may be calibrated, leading to highly accurate calibration of test sample results. As explained hereinabove, these advantages are also applicable to measurable output indications of analytes in the test and control samples other than colorimetric signatures. Colorimetric calibration is referred to hereinabove simply as one preferred embodiment.

In one preferred embodiment of the invention, the control sample colorimetric output may provide a sufficiently accurate calibrating standard with respect to the test sample colorimetric output, such that calibration of the test sample colorimetric output with respect to the control sample colorimetric output may involve a simple qualitative comparison. Such a qualitative comparison may be based on an image processing comparison, such as a machine vision comparison of the colorimetric signatures of the control sample and test sample. For example, machine vision may be used to evaluate whether the color of the test sample colorimetric signature is lighter or darker than the color of the control sample colorimetric signature for one or more analytes of interest. In this case, a single control sample may be used per corresponding test sample. It is appreciated that such qualitative analysis may be quicker and simpler than quantitative colorimetric calibration. A diagnosis of the subject as having diabetes or pre-diabetes may be provided based on such qualitative calibration.

Additionally or alternatively, calibration of the test sample colorimetric signature with respect to the control sample colorimetric signature may involve a quantitative calibration, such as calibration of relative concentrations of analytes based on comparing the colorimetric signatures of the test sample and at least two control samples having mutually different predetermined concentrations of the analyte of interest therein. In this case, the at least two control samples may be used to create a standard curve correlating analyte concentration to colorimetric signature. The concentration of analyte in the test sample may be calibrated with respect to the standard curve.

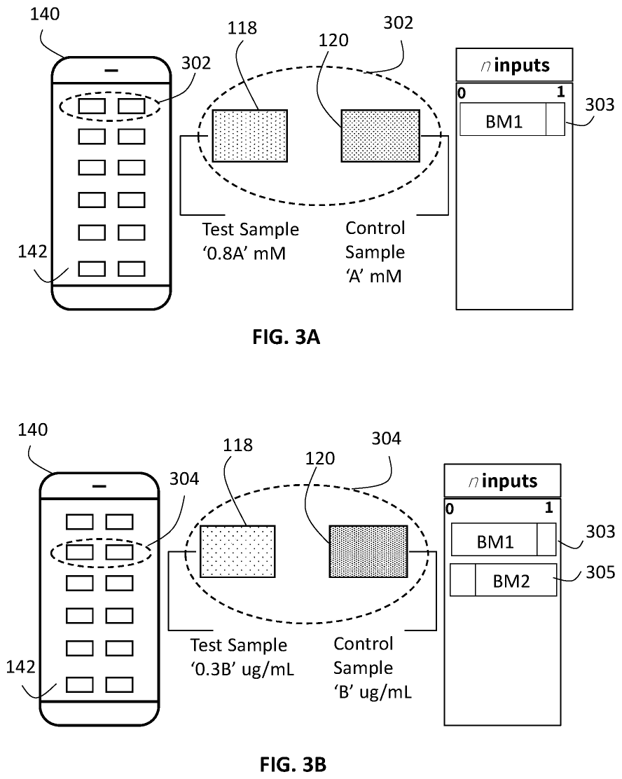

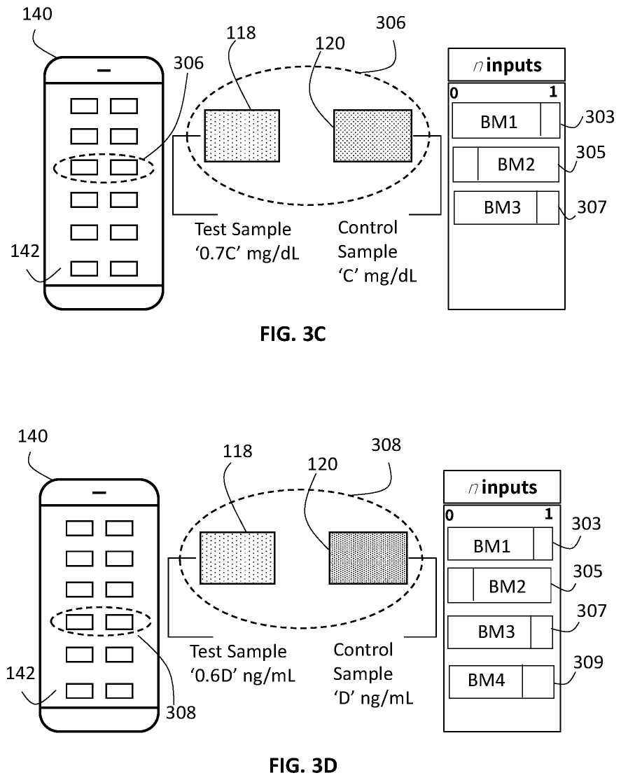

Further details relating to the calibration process are provided henceforth, with reference to FIGS. 3A-3D.

In one preferred embodiment of the present invention, the control sample is processed on the same testing device as that on which the test sample is processed, such that the testing device may be termed ‘self-calibrating’. Thus, the testing, calibrating and diagnosis based thereon is simple, inexpensive and carried out on a compact device, whilst providing highly accurate calibration of the sample colorimetric test results.

As seen in FIG. 1, diagnostic device 102 may be embodied as a diagnostic cartridge, such as a micro-fluidic device in the form of a micro-fluidic plastic chip. Diagnostic device 102 may include a test region, generally indicated by reference number 104 and a control region, generally indicated by reference number 106, which test and control regions 104 and 106 may be generally symmetrical with respect to one another. Test region 104 may refer to a region of device 102 within which a test sample obtained from a subject is processed. The test sample may be any type of bio-fluid, such as saliva, blood or tears by way of example only. Control region 106 may refer to a region of device 102 within which a control sample, such a synthetic fluid manufactured to mimic properties of the test sample for specific analyte thresholds, is processed.

Device 102 preferably includes a sample entry point 110 via which a test sample obtained from a subject may enter device 102. Sample entry point 110 is preferably located within test region 104. It is appreciated that sample entry point 110 is a preferred embodiment of a test sample receipt subsystem operative to receive a test sample from a subject, the test sample possibly containing at least one analyte of interest.

Device 102 further may include a control entry point 112 via which a control may enter device 102. Control entry point 112 is preferably located within control region 106. It is a particular feature of device 102 that the sample is introduced into sample entry point 110 at a time T₁ and the control is introduced into the control entry point 112 at a time equal or almost equal to T₁, such that the control sample and test sample may be processed in parallel, in a temporal sense, within test and control regions 104 and 106. Preferably, the control and test samples are mutually processed within a sufficiently narrow time frame such that the two samples share the same or substantially the same ambient testing conditions.

In one possible embodiment, the control sample is stored within the device and is released at substantially the same time as of the test sample, such that the control sample and test sample may be processed in parallel in a temporal sense.

Test sample entry point 110 and control entry point 112 are preferably respectively connected to a network of tunnels 114 and 116, such as microtunnels, along which the test and control samples respectively travel. Test tunnels 114 are preferably located within test region 104 and control tunnels 116 are preferably located within control region 106. Reagents may be integrated within device 102, such as deposited on the walls of the tunnels 114 and 116, with which reagents the test sample and control sample may mix as they travel therealong.

Tunnels 114 and 116 preferably terminate at a plurality of windows 118 and 120 respectively, through which the colorimetric reactions of the test sample and control are respectively displayed. Windows 118 and 120 may correspond to wells 122 and 124 respectively located therebeneath. Colorimetric reactions of the test sample and control occurring within wells 122 and 124 respectively are preferably visible through windows 118 and 120. It is appreciated that windows 118 and 120 are preferably transparent in order to allow the colorimetric signatures to be visible therethrough. Preferably, each window of the plurality of windows 118 and 120 displays the colorimetric signature of a particular analyte present or possibly present within the control and test samples respectively.

In accordance with a particularly preferred embodiment of the present invention, control sample wells 124 corresponding to windows 120 may be pre-coated with predetermined concentrations of analytes of interest. In this case, the control sample introduced to device 102 may comprise any type of suitable buffer, such as, by way of example, a saline solution. On arrival of the buffer at control wells 124, the buffer may cause resuspension of the analytes deposited in the control wells. Each one of wells 124 may be coated with a single particular analyte of interest.

It is appreciated that tunnels 114 in combination with wells 122 form one embodiment of a first processing subsystem operative to process the test sample in test region 104 of testing device 102. Furthermore, tunnels 116 in combination with wells 124 form one embodiment of a second processing subsystem operative to process, in at least near real time with respect to the processing of the test sample by the first processing subsystem, at least one control sample containing the least one analyte of interest, in control region 106 of testing device 102.

It is appreciated that although a plurality of windows is shown to comprise windows 118 and 120, this is by way of example only. Windows 118 and 120 may each include only a single window or a greater or fewer number of windows, depending on the number of analytes of interest. For example, device 102 may be configured to enable the measurement of approximately 30 different analytes in parallel.

Furthermore, it is appreciated that although a single one of windows 118 and 120 and corresponding wells 122 and 124 therebeneath are shown adjacent one another in each row along device 102, device 102 may be configured with multiple windows 118 and 120 and corresponding wells 122 and 124 in each row thereof. Furthermore, ones of windows 118 and 120 and/or corresponding wells 122 and 124 may be subdivided into multiple regions.

By way of example, control wells 124 may include multiple wells or segregated regions of wells having pre-coated thereon mutually different concentrations of a given analyte of interest. As described hereinabove, the pre-coated analyte may be resuspended upon entry of a control buffer. Colorimetric reactions resulting from different concentrations of the same analyte of interest in control wells 124 may be used to build a standard curve, useful for quantitative calibration of the test sample.

Additionally or alternatively, device 102 may include at least one blank well housing an additional control sample not including any analytes of interest. The colorimetric signature of the blank well may provide a background signature that may be subtracted from the signatures of the control and test samples. It is appreciated, however, that the inclusion of such a blank well may not be required, since in the case of a qualitative comparison being made between the control and test samples, such background subtraction may be unnecessary.

Additionally or alternatively, windows 118 and 120 and corresponding wells 122 and 124 may include enough internal windows and wells for performing duplicates or triplicates, or as many repetitions as desired, in the measurement of each analyte.

It is appreciated that windows 118 form a preferred embodiment of a first data output subsystem operative to output, as a result of processing by the first processing subsystem embodied as tunnels 114 and/or wells 122, a measurable indication of a level of the at least one analyte in the test sample. Furthermore, it is appreciated that windows 120 form a preferred embodiment of a second data output subsystem operative to output, as a result of processing of the control sample, a measurable indication of a level of the at least one analyte in the control sample.

In one possible embodiment of the present invention, the subject to be diagnosed may spit into a container such as a plastic cup. The plastic cup may be equipped with features for sample collection and preparation. The plastic cup may be pre-filled with a control fluid held in a closed compartment within the plastic cup such that the control fluid does not mix with the test sample. The subject may then connect the plastic cup to sample entry point 110 and control entry point 112, thereby releasing the sample and control into respective tunnel networks 114 and 116. For example, the plastic cup may be punctured by sample entry point 110 and control entry point 112, whereby the fluids held therein are released.

In another possible embodiment, the control fluid may be held in a separate blister pack which may be coupled or pre-attached to the control entry point 112. In yet another possible embodiment, the control need not necessarily be introduced externally into the device at the same time as the sample, but rather the device 102 may be pre-filled with a control fluid that is held at or near control entry point 112. Entry of the test sample into device 102 may trigger instant or near-instant release of the control fluid within device 102, such that the two are processed simultaneously within device 102. Other possible release mechanisms and storage mechanisms for the sample and control fluid, as may be apparent to those skilled in the art, are also contemplated.

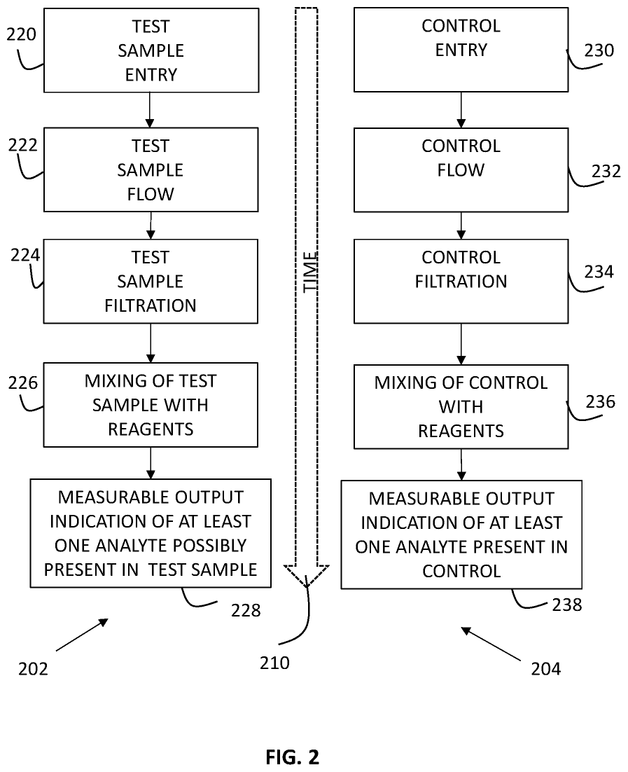

The transition of device 102 from an initial state, in which the test and control samples enter the device, to a subsequent state, in which the test and control samples are processed within the device to generate colorimetric signatures, is indicated by an arrow 126. Further details concerning the passage of the test and control fluids within device 102, and the processing of both therein, are provided henceforth with reference to FIG. 2.

It is appreciated that the structure of device 102 is shown in FIG. 1 in a highly simplified schematic form and that the actual structure of device 102 may be far more complex, including a more intricate network of tunnels as well as additional and/or alternative elements than those shown. It is further appreciated that the configuration of device 102 as a microfluidic chip is by way of example only and that device 102 may be configured in a variety of alternative embodiments, including, by way of example only, as a rapid diagnostic test, lateral flow test, rapid antigen test, test strip, rapid urease test and any type of immunoassay.

Following the flow of the sample and control fluids within device 102, the sample and control fluids preferably respectively arrive at one or more testing chambers, preferably embodied as windows 118 and 120 abutting wells 122 and 124. A chemical reaction preferably occurs within one or more of wells 122, whereby a measurable indication of a level of at least one analyte possibly present in the test sample is generated. Preferably, the chemical reaction involves reaction of the at least one analyte, if present, with an appropriate corresponding at least one reagent in at least one of wells 122. Each type of analyte is preferably reacted with a different given suitable reagent. The reagents are preferably pre-deposited in the wells 122, although in some cases reagents may also or alternatively be pre-deposited in other portions of device 102, such as in the tunnel network thereof.

Simultaneously, partially simultaneously or near simultaneously to the chemical reactions occurring in wells 122, a corresponding chemical reaction preferably occurs within one or more of wells 124, whereby a measurable indication of a level of at least one analyte present in the control sample is generated. Preferably, the chemical reaction involves reaction of the at least one analyte with an appropriate corresponding at least one reagent in at least one of wells 124. Each type of analyte is preferably reacted with a different given suitable reagent. The reagents are preferably pre-deposited in the wells 124, although in some cases reagents may also or alternatively be pre-deposited in other portions of device 102, such as in the tunnel network thereof.

Preferably, the measurable indication of the level of the at least one analyte in the test and control samples is a colorimetric signature corresponding to a level of the analyte in each of the samples. Such colorimetric signatures are indicated in a highly simplified schematic manner by reference number 130.

Preferably, a measurable indication of a level of an individual analyte is generated per testing chamber or window. Further preferably, windows 118 and 120 are arranged such that corresponding analytes are measured beneath corresponding windows and displayed adjacent one another, due to the symmetrical arrangement of test and control regions 104 and 106.

Following the generation of measurable indications of the levels of one or more analytes in the test and control samples and the display thereof on device 102, the measurable indications of the levels of the at least one analyte in the test sample and the control sample are preferably captured by an image acquisition device 140, included in system 100. Image acquisition device 140 may be embodied as any suitable image capture device, such as a smart phone 140. A user of device 102 may use smart phone 140 to take a picture 142 of device 102 and, more particularly, of the region of windows 118 and 120 thereof. The picture 142 of device 102 may be taken using standard photographic capabilities typically present in smart phone 140. Alternatively, a dedicated software or application may be uploaded to smart phone 140 by a user of device 102, which software provides instructions to the user 102 to photograph device 102 and provides a particular platform for doing so. The software may provide instructions to the user regarding a time at which the photograph 142 should be taken following entry of the sample into device 102 and may provide feedback to the user following the taking of photograph 142, for example concerning whether photograph 142 is of acceptable quality, was captured at an appropriate time etc.

Picture 142 may be uploaded from smartphone 140 to a cloud 144. Cloud 144 may include a machine vision algorithm 150 for processing picture 142. Alternatively, machine vision algorithm 150 may be provided by software included in smart phone 140 itself. Machine vision algorithm 150 may be an algorithm employing machine learning for processing picture 142 and more particularly for analyzing the colorimetric signature of the test sample as displayed in one or more of windows 118 with reference to the colorimetric signature of the control sample as displayed in one or more corresponding ones of windows 120.

Machine vision algorithm 150 may be operative to perform a simple qualitative analysis of the colorimetric signature of the test sample, per analyte being detected, with respect to the colorimetric signature of the control sample, per corresponding analyte. By way of example, machine vision algorithm 150 may be operative to classify the test sample colorimetric signature per analyte as less bright, brighter or generally the same as the corresponding control sample colorimetric signature.

Machine vision algorithm 150 may additionally or alternatively be operative to perform a quantitative analysis of the colorimetric signature of the test sample, per analyte being detected, with respect to the colorimetric signature of the control sample, per corresponding analyte. Such a quantitative analysis may involve machine vision algorithm 150 analyzing pixel intensities and/or colors and/or shades displayed in windows 118 and 120 shown in picture 142 so as to derive a concentration of a given analyte in the test and control samples respectively, based on the respective colorimetric signatures thereof. Such a quantitative analysis may use the control sample pixel intensity and/or color and/or shade as related to a concentration of a given analyte in the control sample, in order to calibrate the light spectrum to the analyte concentration and then apply that calibration relationship or scale factor to the light spectrum associated with the same analyte in the test sample.

In one preferred embodiment of the present invention, control windows 120 may display two or more colorimetric signatures respectively corresponding to two or more mutually different predetermined concentrations of a given analyte of interest, in the control sample. By way of example, this may be achieved by pre-coating two or more control wells 124 with mutually different concentrations of a given analyte, which analyte is resuspended upon entry of a control buffer into the coated control wells. It is appreciated that one of the control concentrations of the analyte used to generate the standard curve may be zero or non-zero.

Machine vision algorithm 150 may generate a standard curve by finding a correlation between the at least two different concentrations of the given analyte in the at least two control samples and the colorimetric signatures thereof. The correlation found may subsequently be applied to the colorimetric signature of the analyte in the test sample, in order to derive the concentration thereof.

Irrespective of the particular method employed by machine vision algorithm 150 for processing of the respective colorimetric signatures of the control and test samples, the output of the processing by machine vision algorithm 150 is preferably provided to a data analysis module 160. Data analysis module 160 is preferably operative to receive the processed data from machine vision algorithm 150 and calibrate the test sample colorimetric data with respect to the control sample colorimetric data, in a qualitative and/or quantitative manner. Further details concerning the cooperation between the machine vision algorithm 150 and the calibration functionality of data analysis module 160 are provided henceforth with reference to FIGS. 3A-3D.

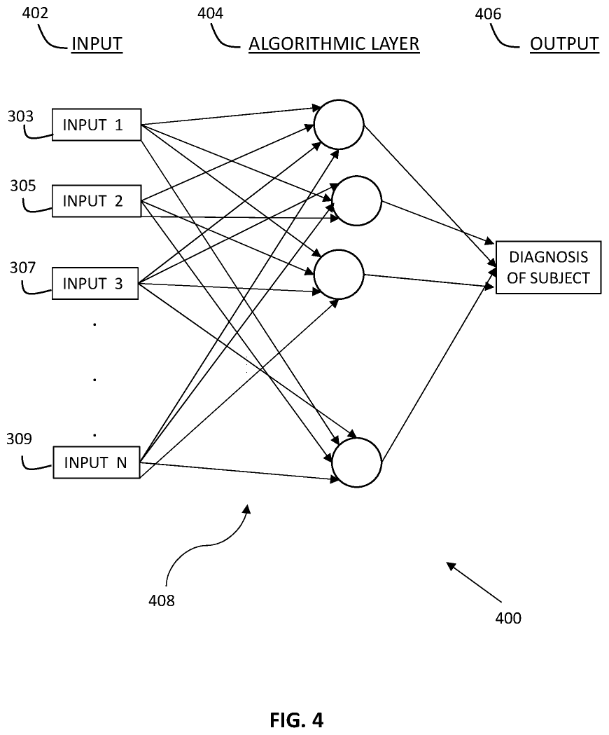

Furthermore, data analysis module 160 is preferably operative to provide a diagnosis of a medical condition or a disposition towards a medical condition of the subject, based on the calibrated test sample data. The diagnosis may be at least one numerical coefficient or index indicative a state of heath of the subject. In some embodiments, the diagnosis may simply be a quantitative measurement or combination of quantitative measurements of one or more of the measured analytes. Particularly preferably, data analysis module is operative to take into account the calibrated test sample data for the one or more analytes of interest and calculate a probability of the subject having a particular medical condition based on a weighted combination of the various calibrated levels of the one or more analytes of interest. A framework for an exemplary algorithm useful for ascertaining such a diagnosis is described in more detail henceforth with reference to FIG. 4.

It is appreciated that although in FIG. 1 data analysis module 160 is shown as a stand-alone module, this is simply for clarity of representation thereof. The functionality of data analysis module 160 may be included in cloud 144. Alternatively, the functionality of data analysis module may be included in device 102 itself. For example, device 102 may include a processor operative to perform data analysis of processed data received from cloud 144. Further alternatively, the functionality of data analysis module 160 may be distributed between cloud 144 and device 102, provided by an external computing device outside of cloud 144 and device 102 and/or combined with the functionality of machine vision algorithm 150.

The output of data analysis module 160 is preferably provided to a user of system 100. The output of data analysis module 160 is preferably in the form of notification 170 of a diagnosis of the subject. Notification 170 may be communicated by data analysis module 160 to a communication device, such as smart phone 140 belonging to the subject or a healthcare provider thereof. By way of example, in the case that data analysis module 160 is included in device 102, a processor in device 102 may be in operative communication with smart phone 140, in order to provide notification 170 thereto. It is understood that notification 170 may take any suitable form known in the art, including a visual, audial or otherwise human sensible output.

Reference is now made to FIG. 2, which is a simplified flow chart illustrating steps in the operation of a diagnostic device of the type shown within the system of FIG. 1.

As described above with reference to FIG. 1, device 102 is preferably operative to process a test sample obtained from a subject in parallel, in a temporal sense, with respect to the processing of a control sample. As seen in FIG. 2, the processing of the test and control samples within device 102 is respectively illustrated in first and second columns 202 and 204. First and second columns 202 and 204 progress along a time axis 210. As appreciated from consideration of the progression of first and second columns 202 and 204, the processing of the test sample and control sample occurs simultaneously in real time or near real time with respect to one another, such that both samples are subject to the same ambient testing conditions and actual control results are available for real time comparison to the test sample results, rather than test sample results being compared to a standardized, less accurate, pre-existing control scale.

Turning to first column 202, processing of the test sample in device 102 is seen to commence at the entry of test sample into device 102 (step 220). Test sample preferably then flows within device 102 (step 222) and may undergo filtration therein (step 224). Test sample preferably undergoes mixing with reagents (step 226), for example with reagents deposited along walls of tunnels 114 and/or within wells 122 (FIG. 1). Following the mixing of the test sample with the reagents, a measurable output indication of at least one analyte possibly present in the test sample is preferably provided (step 228). Such a measurable output indication is preferably in the form of a colorimetric signature generated by the chemical reaction between the one or more analytes of interest in the test sample and corresponding reagents in device 102. The measurable output indication of the at least one analyte may be an output indication of the presence or absence of the analyte and/or an output indication of the level of the analyte.

Turning now to second column 204, processing of the control sample in device 102 is seen to commence at the entry of the control sample into device 102 (step 230). Such entry may involve introduction into device 102 of an external control sample or release of a control sample already held within device 102 for processing therein. Such entry may furthermore comprise entry of a control sample containing at least one predetermined concentration of at least one analyte of interest therein or entry of a blank control sample, such as a saline buffer, functional to subsequently re-suspend at least one predetermined concentration of at least one analyte present within device 102, such as pre-deposited in wells 124. Control sample preferably then flows within device 102 (step 232) and may undergo filtration therein (step 234). Control sample preferably undergoes mixing with reagents (step 236), for example with reagents deposited along walls of tunnels 116 and/or in wells 124 (FIG. 1).

Following the mixing of the test sample with the reagents, a measurable output indication of at least one analyte present in the control sample is preferably provided (step 238). Such a measurable output indication is preferably in the form of a colorimetric signature generated by the chemical reaction between the one or more analytes of interest in the control sample and corresponding reagents in device 102. The measurable output indication of the at least one analyte may be an output indication of the presence or absence of the analyte and/or an output indication of at least one level of the analyte.

It is appreciated that the steps shown in columns 202 and 204 are exemplary only and that these steps may be obviated if unnecessary, supplemented by additional and/or alternative steps, as well as performed in a different order to that shown. It is understood that the performance of the steps shown in columns 202 and 204 is one preferred embodiment of the transition between states of device 102 indicated by arrow 126 in FIG. 1.

Reference is now made to FIGS. 3A-3D, which are simplified exemplary outputs of a diagnostic device of the type of FIGS. 1 and 2.

As described above with reference to FIG. 1, photograph 142 is taken, for example by smart phone 140, of the colorimetric signatures of the test and control samples displayed in reaction windows 118 and 120. The colorimetric signatures shown in photograph 142 are processed by machine vision algorithm 150, either within smart phone 140 and/or in the cloud 144. The processed colorimetric signatures are then provided to data analysis module 160 for relative calibration and diagnosis generation.

The cooperation between the functionalities of machine vision algorithm 150 and data analysis module 160 is shown in FIGS. 3A-3D. It is understood that although FIGS. 3A-3D illustrate a calibration method that involves quantitative calibration, this is not necessarily the case, and a simpler qualitative calibration approach may additionally or alternatively employed. Furthermore, it is understood that FIGS. 3A-3D illustrate the calibration of four analytes or biomarkers, whereas in actuality more or fewer analytes may be calibrated, depending on the testing requirements.

Turning first to FIG. 3A, measurable indications of a first analyte are displayed in a region 302 of photograph 142 and are additionally shown in an enlarged view for clarity thereof. Here, by way of example, the first analyte is indicated to be biomarker 1 (‘BM1’). A known, pre-determined concentration of BM1 in the control sample is indicated in FIG. 3A as ‘A’ mM. Machine vision processing of window 120 in region 302 may ascertain that the control sample has a particular colorimetric signature corresponding to concentration ‘A’ mM.

Machine vision processing of corresponding window 118 in region 302 may ascertain a colorimetric signature of the test sample, as visible through window 118. For example, as shown here, the colorimetric signature of the test sample may correspond to a concentration of ‘0.8A’ mM of BM1. Relative concentrations of BM1 in the saliva and control are represented here very loosely by patterns of mutually different densities, for illustrative purposes only.

It is appreciated that although only a single control window 120 is shown in FIG. 3A, more than one control window may be possible. For example, an absolute concentration of BM1 in the test sample may be found by generating a standard curve based on colorimetric signatures generated by at least two different predetermined concentrations of BM1 in at least two control samples. The concentration of BM1 in the test sample may be found with respect to such a standard curve. Alternatively, a characteristic of the colorimetric signature of the test sample, such as pixel intensity, may simply be defined quantitatively relative to a corresponding characteristic of the colorimetric signature of the control sample, but without necessarily ascertaining the corresponding absolute concentration of BM1 in the test sample. In this latter case, a concentration of BM1 of 0.8A mM in the test sample is not to be understood as an absolute concentration but rather a relative rating of a colorimetric signature of the test sample with respect to a colorimetric signature of the control sample corresponding to a concentration of ‘A’ mM.

The concentration of BM1 in saliva may be calibrated with respect to the concentration of BM1 in the control, for example by data analysis module 160. For example, the concentration of BM1 in the saliva may be rated on a scale from 0 to 1 relative to the concentration of BM1 in the control. In the example shown, a rating of 0.8, indicated by a reference number 303, is assigned to express the calibrated BM1 concentration in the saliva.

Turning now to FIGS. 3B-3D, measurable indications of a second analyte are displayed in a region 304 of photograph 142 (FIG. 3B), a third analyte in a region 306 (FIG. 3C) and a fourth analyte in a region 308 (FIG. 3D), all additionally shown in enlarged views in the respective drawings, for clarity of presentation. Here, by way of example, the second analyte shown in FIG. 3B is indicated to be BM2, the third analyte shown in FIG. 3C is indicated to be BM3 and the fourth analyte shown in FIG. 3D is indicated to be BM4.

Machine vision processing of window 120 in region 304 in FIG. 3B may ascertain a colorimetric signature of the control sample, here a synthetic fluid by way of example, corresponding to a known predetermined concentration of ‘B’ ug/mL of BM2. Machine vision processing of corresponding window 118 in region 304 may ascertain a colorimetric signature of the test sample, here saliva by way of example. For example, the colorimetric signature may correspond to a concentration of BM2 in the test sample, of ‘0.3B’ ug/mL. The relative concentrations of BM2 in the saliva and control are loosely represented here by patterns of mutually different densities, for illustrative purposes only. As detailed hereinabove with respect to FIG. 3A, the absolute concentration of BM2 in the test sample may be found or a relative concentration of BM2 in the test sample may be found based on quantitative comparison of the colorimetric signatures of the test and control samples.

The concentration of BM2 in saliva may be calibrated with respect to the concentration of BM2 in the control, for example by data analysis module 160. For example, the concentration of BM2 in the saliva may be rated on a scale from 0 to 1 relative to the concentration of BM2 in the control. In the example shown, the concentration of BM2 in the saliva is considerably less than the concentration of BM2 in the control and a rating of 0.3, indicated by a reference number 305, is assigned to express the calibrated BM2 concentration in the saliva.

Machine vision processing of window 120 in region 306 in FIG. 3C may ascertain a colorimetric signature of the control sample, here a synthetic fluid by way of example, corresponding to a known predetermined concentration of ‘C’ mg/dL of BM3. Machine vision processing of corresponding window 118 in region 306 may ascertain a colorimetric signature of the test sample, here saliva by way of example. For example, the colorimetric signature may correspond to a concentration of BM3 in the test sample, of ‘0.7C’ mg/dL. The relative concentrations of BM3 in the saliva and control are loosely represented here by patterns of mutually different densities, for illustrative purposes only. As detailed hereinabove with respect to FIG. 3A, the absolute concentration of BM3 in the test sample may be found or a relative concentration of BM3 in the test sample may be found based on quantitative comparison of the colorimetric signatures of the test and control samples.

The concentration of BM3 in saliva may be calibrated with respect to the concentration of BM3 in the control, for example by data analysis module 160. For example, the concentration of BM3 in the saliva may be rated on a scale from 0 to 1 relative to the concentration of BM3 in the control. In the example shown, the concentration of BM3 in the saliva is somewhat less than the concentration of BM3 in the control and a rating of 0.7, indicated by a reference number 307, is assigned to express the calibrated BM3 concentration in the saliva.

Machine vision processing of window 120 in region 308 in FIG. 3D may ascertain a colorimetric signature of the control sample, here a synthetic fluid by way of example, corresponding to a known predetermined concentration of ‘D’ ng/mL of BM4. Machine vision processing of corresponding window 118 in region 308 may ascertain a colorimetric signature of the test sample, here saliva by way of example. For example, the colorimetric signature may correspond to a concentration of BM4 in the test sample, of ‘0.6C’ mg/dL. The relative concentrations of BM4 in the saliva and control are loosely represented here by patterns of mutually different densities, for illustrative purposes only. As detailed hereinabove with respect to FIG. 3A, the absolute concentration of BM4 in the test sample may be found or a relative concentration of BM4 in the test sample may be found based on quantitative comparison of the colorimetric signatures of the test and control samples.

The concentration of BM4 in saliva may be calibrated with respect to the concentration of BM4 in the control, for example by data analysis module 160. For example, the concentration of BM4 in the saliva may be rated on a scale from 0 to 1 relative to the concentration of BM4 in the control. In the example shown, the concentration of BM4 in the saliva is less than the concentration of BM4 in the control and a rating of 0.6, indicated by a reference number 309, is assigned to express the calibrated BM4 concentration in the saliva.

The ratings 303, 305, 307 and 309, of the various analytes may be combined, for example by data analysis module 160, in order to arrive at a diagnosis of the subject from which the specimen was obtained. A possible approach for the combination for the ratings of the various analytes is shown in FIG. 4.

As seen in FIG. 4, an algorithm 400 may be applied to the ratings in order to arrive at an output relating to a state of health of the subject from which the test sample was obtained. The output may be a diagnosis, including, by way of example only, an index related to or indicative of a diagnosis, or an indication of a likelihood of a current or impending diagnosis. In some embodiments, the diagnosis may simply be a quantitative measurement or combination of quantitative measurements of one or more of the measured analytes. Algorithm 400 may include an input layer 402, an algorithmic processing layer 404 and an output layer 406.

Algorithmic layer 404 may be a machine learned layer that is operative to weigh the various ratings with respect to one another and to derive a combined weighted sum expressing the combined diagnostic significance of the various weightings.

Algorithm 400 may receive at input layer 402 the ratings 303, 305, 307 and 309. A multiplicity of arrows 408 extending between the input layer 402 and algorithmic layer 404 and between the algorithmic layer 404 and output 406 symbolically represent mathematical functions forming a part of algorithm 400. Each mathematical function as represented by a single one of arrows 408 preferably has an associated threshold, weighting value and activation value, determining under what conditions the given function is activated and how. The threshold, weighting and activation values of each function, as well as the functions themselves, are preferably found and set as part of a learning process carried out by algorithm 400. It is understood that the mathematical functions represented by arrows 506 are depicted in a highly simplified symbolic manner in FIG. 4 in order to represent the general arrangement thereof.

Algorithm 400 may involve a single layer of a learning process between the input and output stages, as illustrated in FIG. 4. Alternatively, algorithm 400 may involve deep learning in which multiple layers of a learning process are present between the input and output stages, which multiple layers may, but do not necessarily, increase the accuracy of the learning process.

Following the weighted combination of the various inputs within algorithmic layer 404, an output is provided at output layer 406, in the form of a diagnosis. The diagnosis may be expressed as binary output, indicating the presence or absence of a particular condition, or as a probability that the subject presently has, or is inclined to develop, one or more possible medical conditions.

The weighted sum algorithm executed by layer 404 may take a large variety of appropriate forms, as are well known in the art.

The weighted sum algorithm executed by algorithmic processing layer 404 per individual input may be expressed as:

$${A}_{i} = {\sum }_{j}^{n} {w}_{ij} {I}_{j}^{{α}_{j}} , i = 1 , 2 , 3 , … , k$$

where Iⱼ is the analyte input rating, such as one of ratings 303, 305, 307 and 309, wᵢⱼ is a relative weight assigned to the particular analyte, and αⱼ is an exponential power set per analyte.

The weighted sum algorithm per input may be iteratively combined across all of the inputs in accordance with

$${B}_{i} = {\sum }_{j}^{k} {u}_{ij} {A}_{j}^{{β}_{j}} , i = 1 , 2 , 3 , … , v$$

where uᵢⱼ is the relative weight, Aⱼ is the value of the iteration, and βⱼ is an exponential power set per iteration. Bi is an expression of the weighted sum of the all of the iterations across all of the input analytes, which yields the output diagnosis 408. The values of the variables in the equations above may be fixed based on a training data set or may be dynamically ascertained during the course of testing.

It is appreciated that the weighted sum algorithm may be applied as a machine learned algorithm, in which the values of the various parameters are set through machine learning, or may be applied as a simple mathematical operation in which the values of the various parameters are pre-set based on reference values as may be known in the art.

It is appreciated that the particular embodiment of algorithm 400 illustrated and described herein is provided by way of example only and that a wide variety of other algorithms, employing machine learning or other techniques, may be utilized in various embodiments of the present invention in order to analyze the calibrated analyte indications in the test sample.

It is appreciated that the ratings of the various biomarkers in the saliva are calibrated with respect to the measured levels of those biomarkers in the control fluid. Should the control fluid not be processed per saliva sample, simultaneously with respect thereto as is carried out in accordance with preferred embodiments of the present invention, the accuracy of the calibration would be diminished and the subsequent diagnosis rendered less accurate.

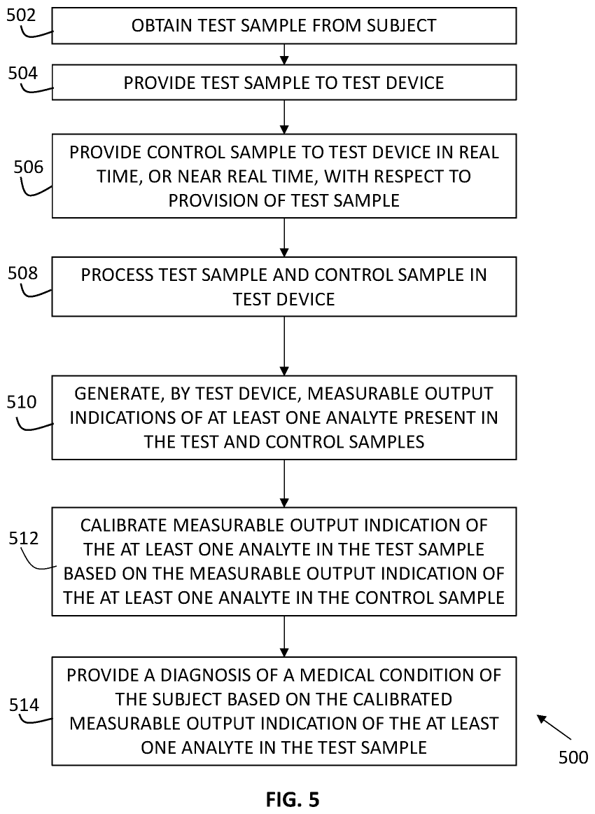

Reference is now made to FIG. 5, which is is a simplified flow chart diagram illustrating a method for obtaining calibrated indicia of a level of an analyte in a sample, in accordance with another preferred embodiment of the present invention.

As seen in FIG. 5, a method 500 may begin at a first step 502 at which a test sample is obtained from a subject. The test sample is preferably provided to a test device, as seen at a second step 504. An additional, separate, control sample is preferably provided to the test device in real time, or near real time, with respect to the provision of the test sample, as seen at a third step 506. It is appreciated that the control sample may be provided automatically upon provision of the test sample, for example by entry of the test sample triggering automatic release of the control sample.

The test and control samples are preferably processed within the test device, as seen at a fourth step 508. The test and control samples are particularly preferably simultaneously, or near simultaneously, processed within the test device. As seen at a fifth step 510, measurable output indications of at least one analyte present in the control sample, and possibly present in the test sample, are preferably generated by the test device. Particularly preferably, the measurable output indications are in the form of optically measurable measurements, such as colorimetric signatures involving one or more of varying color frequencies, intensities and amplitudes. The at least one analyte may be contained in the control sample upon entry of the control sample. Alternatively, the at least one analyte may not be present in the control sample upon entry of the control sample, but rather may be added to the control sample thereafter. By way of example, the control sample may be a buffer and analytes may be resuspended upon contact therewith, following which such analytes may be detected.

As seen at a sixth step 512, the measurable output indication of the at least one analyte in the test sample is preferably calibrated with respect to the measurable output indication of the at least one analyte in the control sample. Such calibration may be quantitative, qualitative or both. In the case of a quantitative analysis, such analysis may involve the generation of a standard curve based on two or more measurable output indications of two or more respective concentrations of the at least one analyte in the control sample. Additionally, or alternatively, in the case of a quantitative analysis, the calibration may involve subtraction of a background measurable indication associated with at least one control sample not including the analyte. It is understood that such a background measurable indication may constitute one of the measurable indications based upon which the standard curve is generated or may be in addition thereto.

As seen a seventh step 514, the calibrated measurable output indication of the at least one analyte in the test sample is then used as a basis for providing a diagnosis of a medical condition of the subject from whom the test sample was obtained. In the case of multiple analytes having been measured, the calibrated measurable output indications of the multiple analytes may be combined, preferably although not necessarily in a weighted manner, in order to arrive at the diagnosis.

It will be appreciated by persons skilled in the art that the present invention is not limited by what has been particularly claimed hereinbelow. Rather, the scope of the invention includes various combinations and subcombinations of the features described hereinabove as well as modifications and variations thereof as would occur to persons skilled in the art upon reading the forgoing description with reference to the drawings and which are not in the prior art.

1. A method for obtaining calibrated indicia of a level of at least one analyte in a sample comprising: introducing a test sample possibly containing at least one analyte into a testing device; processing said test sample in a test region of said testing device; obtaining, as a result of said processing, a measurable indication of a level of said at least one analyte in said test sample; processing at least one control sample containing said at least one analyte in a control region of said testing device, said processing of said at least one control sample being carried out in at least near real time with respect to said processing of said test sample; obtaining, as a result of said processing of said control sample, at least one measurable indication of at least one level of said at least one analyte in said at least one control sample; and calibrating said indication of said level of said at least one analyte in said test sample based on said at least one measurable indication of said level of said at least one analyte in said at least one control sample, to provide a calibrated indication of said level of said at least one analyte in said test sample.

2. A method according to claim 1, wherein said control sample is separate to and different from said test sample.

3. A method according to claim 1, wherein said test sample comprises a bodily fluid obtained from a subject and said testing device is a single-use, disposable device, usable by said subject.

4. (canceled)

5. A method according to claim 1, wherein said measurable indication of said level of said at least one analyte in said test sample and said control sample comprises at least one of a colorimetric indication, an optical indication, an electrical indication and a chemical indication.

6. A method according to claim 5, wherein said calibrating comprises at least one of: qualitative calibrating based on relative characteristics of said measurable indications of said test sample and control sample, and quantitative calibrating based on relative concentrations of said at least one analyte as derived from said measurable indications of said test sample and control sample.

7. A method according to claim 6, wherein: said at least one control sample comprises at least two control samples having mutually different concentrations of said analyte therein; and said quantitative calibrating comprises finding a correlation between said different concentrations of said analyte in said at least two control samples and said measurable indications thereof, and applying said correlation to said measurable indication of said level of said analyte in said test sample.

8. A method according to claim 6, and also comprising, following said calibrating: rating a level of said at least one analyte in said test sample based on said calibrating, and providing a diagnosis of a subject from whom said test sample is obtained based on said rated level of said at least one analyte in said test sample.

9. A method according to claim 8, wherein said providing a diagnosis comprises providing one of a binary diagnosis and a probability of said subject having said diagnosis.

10. A method according to claim 1, wherein said calibrating is at least partially carried out by processing functionality within at least one of said testing device and the cloud.

11-20. (canceled)

21. A testing device comprising: a test region configured to process therein a test sample obtained from a subject; a first output region configured to display a measurable indication of a level of at least one analyte in said test sample; a control region configured to process therein at least one control sample, said control sample being processed in at least near real time with respect to said test sample; and a second output region configured to display, in at least near real time with respect to said display of said measurable indication of said level of said at least one analyte in said test sample, a measurable indication of at least one level of said at least one analyte in said at least one control sample, said measurable indication of said at least one level of said at least one analyte in said at least one control sample providing a basis for calibration of said measurable indication of said level of said at least one analyte in said test sample.

22-30. (canceled)

31. A system for diagnosing a subject comprising: a testing device comprising: a test sample receipt subsystem operative to receive a test sample from a subject, said test sample possibly containing at least one analyte; a first processing subsystem operative to process said test sample in a test region of said testing device; a first data output subsystem operative to output, as a result of said processing by said first processing subsystem, a measurable indication of a level of said at least one analyte in said test sample; a second processing subsystem operative to process, in at least near real time with respect to said processing of said test sample by said first processing subsystem, at least one control sample containing said least one analyte, in a control region of said testing device; and a second data output subsystem operative to output, as a result of said processing of said control sample, a measurable indication of at least one level of said at least one analyte in said at least one control sample, an image acquisition device operative to capture said measurable indications of said levels of said at least one analyte in said test sample and said at least one control sample, and a data analysis module operative to calibrate said captured measurable indication of said level of said at least one analyte in said test sample based on said captured measurable indication of said at least one level of said at least one analyte in said at least one control sample and to output a diagnosis of said subject based on said calibrated level of said at least one analyte in said test sample.

32. A system according to claim 31, wherein said control sample is separate to and different from said test sample.

33. A system according to claim 31, wherein said test sample comprises a bodily fluid obtained from said subject.

34. A system according to claim 31, wherein said testing device is a single-use, disposable device.

35. A system according to claim 31, wherein said measurable indication of said level of said at least one analyte in said test sample and said control sample comprises at least one of a colorimetric indication, an optical indication, an electrical indication and a chemical indication.

36. A system according to claim 35, wherein said data analysis module is operative to perform at least one of: qualitative calibration based on relative characteristics of said measurable indications of said test sample and control sample, and quantitative calibration based on relative concentrations of said at least one analyte as derived from said measurable indications of said test sample and control sample.

37. A system according to claim 36, wherein: said at least one control sample comprises at least two control samples having mutually different concentrations of said analyte therein; and said data analysis module is operative to find a correlation between said different concentrations of said analyte in said at least two control samples and said measurable indications thereof, and to apply said correlation to said measurable indication of said level of said analyte in said test sample in order to derive said quantitative calibration.

38. A system according to claim 36, wherein said data analysis module is also operative to: rate a level of said at least one analyte in said test sample based on said calibration, and provide said diagnosis of said subject from whom said test sample is obtained based on said rated level of said at least one analyte in said test sample.

39. A system according to claim 37, wherein said diagnosis comprises one of a binary diagnosis and a probability of said subject having said diagnosis.

40. A system according to claim 31, wherein said data analysis module is at least partially incorporated within at least one of said testing device and processing functionality in the cloud.
